(19) United States
(12) Patent Application Publication
(10) Pub. No.: US 2008/0281199 A1
(43) Pub. Date: Nov. 13, 2008

(54) SYSTEM AND METHOD FOR A DUAL SHOCK SOURCE LITHOTRIPSY SYSTEM

(75) Inventors: Steven DeBrock, Roswell, GA (US); Cengiz Kabakci, Ankara (TR)

Correspondence Address: DUBOIS, BRYANT, CAMPBELL & SCHWARTZ, LLP, 700 LAVACA STREET, SUITE 1300, AUSTIN, TX, 78701, US

(73) Assignee: HealthTronics, Inc., Austin, TX (US)

(21) Appl. No.: 11/746,379

(22) Filed: May 9, 2007

(51) Int. Cl. A61B 17/225

(52) U.S. Cl. 600/439

(57) ABSTRACT

A system and method is provided for presenting a patient with lithotripsy medical treatments. The patient is placed upon a patient table for a lithotripsy treatment such that a region of the patient's body may be targeted with the lithotripsy system. The lithotripsy system may include either a first therapy head or a second therapy head. The therapy head included in the system is moveably coupled within the lithotripsy system to provide fine adjustments to shockwave projections of the therapy head. The therapy head may be adjusted such that a specific target area in a patient treatment region may be locked in for shockwave treatment. Other embodiments are also disclosed.

### BACKGROUND OF THE INVENTION

1. Technical Field

The present invention relates in general to a system and method for a dual shock source lithotripter. In particular, the present invention relates to a system and method that encompasses both electro-hydraulic and electromagnetic shock sources.

2. Description of the Related Art

Lithotripter technology is used in association with devices that destroy kidney stones. For example, kidney stones are fragmented by passing shock waves through water-filled therapy heads. The procedure creates stone fragments small enough to be expelled in the urine of the patient.

In other words, a lithotriptor is a medical device used in the non-invasive treatment of kidney stones (urinary calculosis), various orthopedic applications, and biliary calculi (stones in the gallbladder or in the liver). The scientific name of this procedure is Extracorporeal Shock Wave Lithotripsy (ESWL).

Common lithotripter devices include either an electro-hydraulic shock source or an electromagnetic shock source. The shock source includes a device known as a therapy head to perform the kidney stone fragmentation procedure. Interchangeability of different therapy heads is not currently available with lithotripter devices because such therapy heads require precise alignment to establish appropriate mounting connections. For example, because capacitance requirements of an electromagnetic therapy head greatly exceeds the capacitance requirements of an electro-hydraulic therapy head shock source, the therapy head of a lithotripter device is not interchangeable between an electromagnetic therapy head and an electro-hydraulic therapy head. Further, tolerance levels of lithotripter devices do not account for exchanging therapy heads, nor are lithotripter devices designed to accommodate the different operational aspects which may be encountered by the different types of therapy heads that are currently available with lithotripter devices.

Many other problems and disadvantages of the prior art will become apparent to one skilled in the art after comparing such prior art with the present invention as described herein.

### BRIEF DESCRIPTION OF TILE DRAWINGS

The present invention may be better understood, and its numerous objects, features, and advantages made apparent to those skilled in the art by referencing the accompanying drawings.

FIG. 1 is a diagram showing a perspective view of a general lithotripsy system constructed according to principles of one embodiment of the present invention;

FIG. 2 is a diagram showing an embodiment of a first therapy head that may be used by the lithotripsy system of FIG. 1;

FIG. 3 is a diagram showing an embodiment of a second therapy head that may be used by the lithotripsy system of FIG. 1;

FIG. 4 is a diagram showing an embodiment of a mounting configuration for mounting a therapy head onto the lithotripsy system of FIG. 1;

FIG. 5A is a diagram showing a front view of an embodiment of a lithotripsy system having a c-arm;

FIG. 5B is a diagram showing a side view of an embodiment of the lithotripsy system and c-arm of FIG. 5A; and

FIG. 5C is a diagram showing a perspective view of an embodiment of the lithotripsy system and c-arm of FIG. 5A.

### DETAILED DESCRIPTION OF THE DRAWINGS

It has been discovered that the aforementioned challenges are resolved using a system and method that allows the interchanging of therapy heads on lithotipter devices. The following is intended to provide a detailed description of an example of the invention and should not be taken to be limiting of the invention itself. Rather, any number of variations may fall within the scope of the invention, which is defined in the claims following the description.

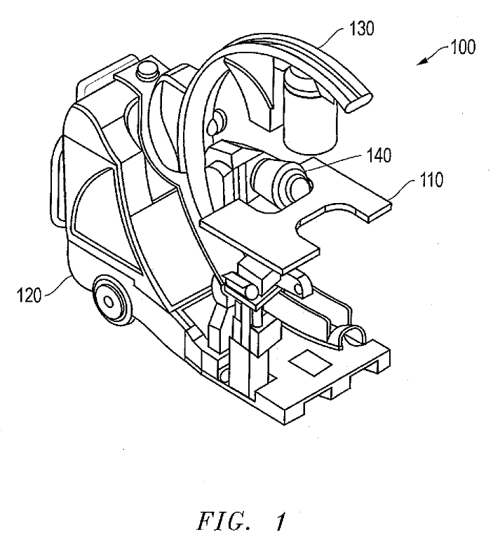

FIG. 1 is a diagram showing a perspective view of a general lithotripsy system 100 constructed according to principles of one embodiment of the present invention. Lithotripsy system 100 includes a patient table 110 that is movably attached to a mobile cart 120. Also movably attached to mobile cart 120 is a c-arm 130 that may be manipulated about a patient that is positioned on patient table 110.

A therapy head 140 is also attached to c-arm 130 wherein fine tune adjustments may be performed with therapy head 140. The fine tune adjustments allow therapy head 140 to locate a particular target in a patient treatment region. The patient treatment region is a portion of the patient's body that is receiving lithotripsy treatment on patient table 110.

Lithotripsy treatment is an attempt to break up a stone (such as a kidney stone) in a patient. This breaking up of the kidney stone is performed with minimal collateral damage to the patient. Lithotripsy's use of an externally-applied, focused high-intensity acoustic pulse is the guiding principle for such treatment.

An anesthetized patient lies down on, for example, patient table 110 with their back supported by a water-filled coupling device placed at the patient kidney level, for instance. A fluoroscopic x-ray imaging system or an ultrasound imaging system is used to locate the stone and aim therapy head 140 such that a shock wave is focused upon the offending stone.

The treatment usually starts at a lithotripsy system's lowest power level. Treatment continues with a long gap between shock wave pulses in order to accustom the patient to the sensation. In order to break up the stone more effectively, the frequency of pulses and power level then gradually increases. The final power level usually depends on the patient's pain threshold. If the stone is positioned near a bone (usually a rib in the case of kidney stones), then the lithotripsy treatment may be more uncomfortable as the shock waves can cause a mild resonance in the bone which can be felt by the patient.

Upon fragmentation of a kidney stone, the smaller pieces of the stone can be flushed from the patient's body. For example, a ureteral stent (a kind of expandable hollow tube) may be used to pass the fragmented stone from the patient. The process takes about an hour.

Therapy head 140 is constructed such that other therapy heads may be used to easily replace therapy head 140 and to perform other types of lithotripsy treatments with lithotripsy system 100. For example, lithotripsy system 100 may be used to perform either electromagnetic or electro-hydraulic lithotripsy. The choice depends upon the therapy head that is selected for the lithotripsy treatment.

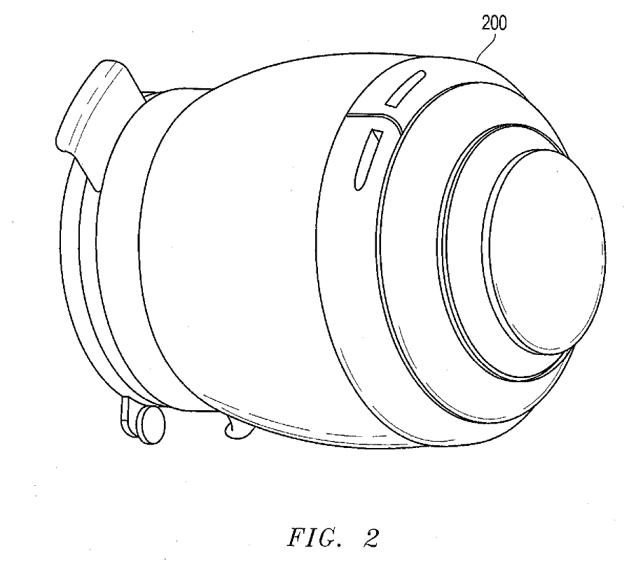

FIG. 2 is a diagram showing an embodiment of a first therapy head 200 that may be used by lithotripsy system 100. First therapy head 200 is constructed with precise machining that supports rapid replacement with therapy head 140 through detachment and/or re-attachment of therapy head 140 in lithotripsy system 100. Therapy heads such as first therapy head 200 are conducive to electromagnetic shock wave therapy because tolerance requirements of lithotripsy system 100 have been met by therapy heads 140 and 200.

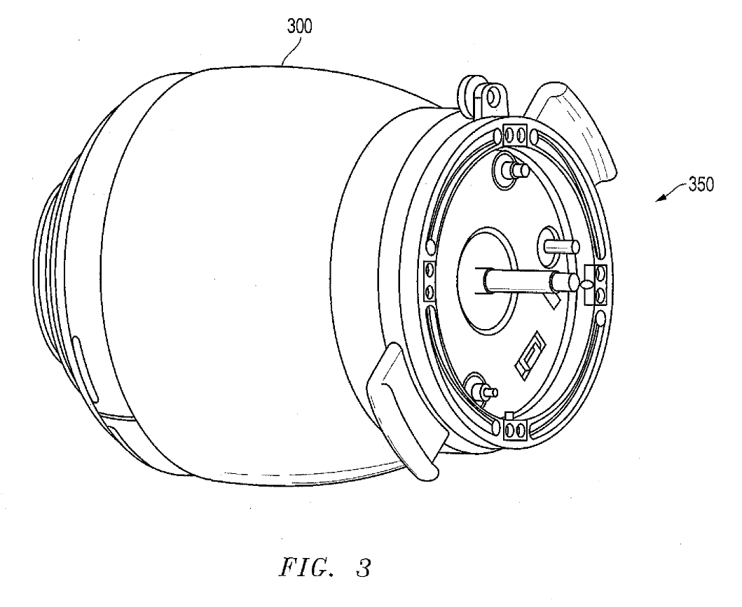

FIG. 3 is a diagram showing an embodiment of a second therapy head 300 that may be used by lithotripsy system 100. Similar to first therapy head 200, second therapy head 300 is constructed with precise machining that supports rapid replacement of therapy head 140 in lithotripsy system 100. However, therapy heads such as second therapy head 300 are conducive to electro-hydraulic shock wave therapy because tolerance requirements of lithotripsy system 100 have been met by second therapy head 300. Specifically, as illustrated, second therapy head 300 includes hydraulic coupling hardware 350 that meets the precise machining requirements of lithotripsy system 100. Similar to first therapy head 200, detachment and re-attachment of second therapy head 300 is enabled by the precise machining and specialized circuitry of the therapy heads.

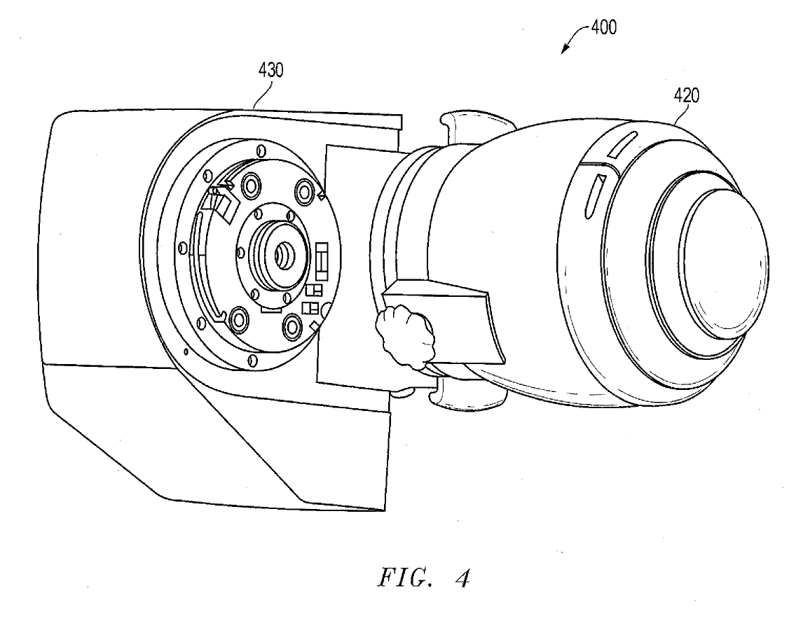

FIG. 4 is a diagram showing an embodiment of a mounting configuration 400 for mounting a therapy head 420 onto mounting bracket 430 of a lithotripsy system such as lithotripsy system 100. Mounting bracket 430 includes special circuitry that has been designed to meet the high voltage requirements necessary to produce an acoustic shock wave from therapy head 420. In at least one embodiment, in line with conventional use, therapy head 420 generates a shock wave which is directed onto a specific target on a patient thereby allowing the lithotripsy system to operate therapeutically.

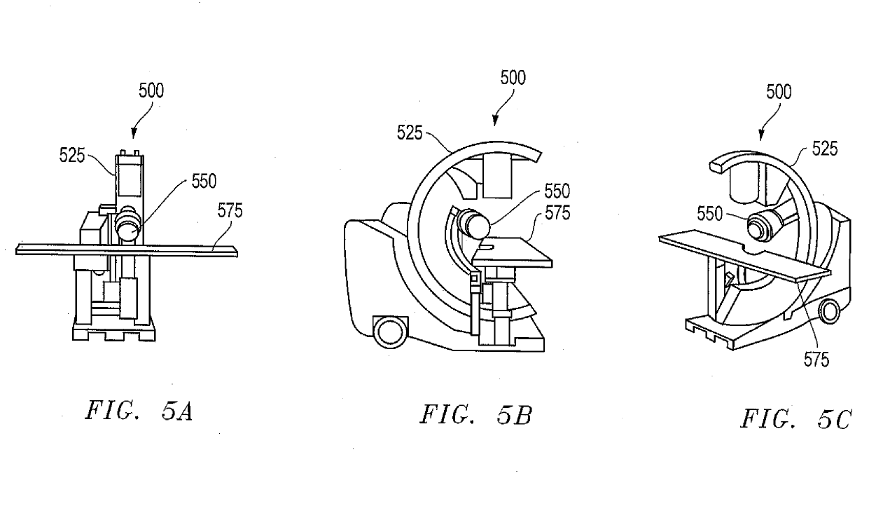

FIG. 5A is a diagram showing a front view of an embodiment of a lithotripsy system 500 having a c-arm 525. C-arm 525 assists in the positioning of a therapy head 550 with respect to a patient positioned on patient table 575. Likewise, FIGS. 5B and 5C are diagrams showing a side view and a perspective view, respectively, of lithotripsy system 500, c-arm 525, therapy head 550, and patient table 575. Lithotripsy system 500 is designed such that patient table 575 is used to position the body of a patient for medical treatment directed toward a single portion of the patient's body, such as a kidney stone, gallstone, various orthopedic applications, and so forth. The medical treatment is provided through the use of the lithotripsy system 500.

Depending on the medical treatment technique that is selected for the patient, a first therapy head is selected to be moveably coupled within the lithotripsy system 500 as illustrated by therapy head 550. Therapy head 550 may be selected from the group of an electromagnetic therapy head, an electro-hydraulic therapy head, or some other type of therapy head that is designed to provide the desired therapeutic treatment for a patient.

Therapy head 550 is designed to provide fine adjustments to shockwave propagation direction from the therapy head 550 such that a specific target area in a patient treatment region may be located for shockwave treatment. A second therapy head is available to interchangeably replace or be exchanged with the first therapy head within lithotripsy system 500.

The foregoing is provided simply to demonstrate at least one example of a preferred embodiment according to principles of the present invention and thus contains, by necessity, simplifications, generalizations, and omissions of detail; consequently, those skilled in the art will appreciate that the foregoing disclosure is illustrative only and is not intended to be in any way limiting. Other aspects, inventive features, and advantages of the present invention, as defined solely by the claims, will become apparent to those of ordinary skill in the art upon viewing the non-limiting detailed description.

One of the preferred implementations of the invention includes a software program, namely, a set of instructions (program code) or other functional descriptive material in a code module that may, for example, be resident in a random access memory of a computer. Until required by the computer, the set of instructions may be stored in another computer memory, for example, in a hard disk drive, or in a removable memory such as an optical disk (for eventual use in a CD ROM) or floppy disk (for eventual use in a floppy disk drive), or downloaded via the Internet or other computer network. Thus, embodiments of the present invention may be implemented as a computer program product for use in a computer.

In addition, although the various methods described are conveniently implemented in a general purpose computer selectively activated or reconfigured by software, one of ordinary skill in the art would also recognize that such methods may be carried out in hardware, in firmware, or in more specialized apparatus constructed to perform the required method steps. Functional descriptive material is information that imparts functionality to a machine. Functional descriptive material includes, but is not limited to, computer programs, instructions, rules, facts, definitions of computable functions, objects, and data structures.

While particular embodiments of the present invention have been shown and described, those skilled in the art will appreciate that, based upon the teachings herein, changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims.

It will be understood by those with skill in the art that if a specific number of an introduced claim element is intended, such intent will be explicitly recited in the claim, and in the absence of such recitation no such limitation is present. For non-limiting examples, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A lithotripsy system comprising:
a patient table for positioning a patient body for medical treatment in a region of the patient body using the lithotripsy system;
a first therapy head moveably coupled within the lithotripsy system, the first therapy head moveably coupled to provide fine adjustments to the first therapy head such that a specific target area of the patient treatment region may be located for shockwave treatment using a first technique conducive to the first therapy head; and
a second therapy head interchangeably configured to be exchangeable with the first therapy head within the lithotripsy system, wherein the second therapy head is configured to provide shockwave treatment using a second technique.

2. The lithotripsy system of claim 1 wherein the first therapy head is configured to perform a first shockwave treatment distinct from a second shockwave treatment of the second therapy head.

3. The lithotripsy system of claim 1 wherein the second therapy head is configured to enable rapid exchanging with the first therapy head.

4. The lithotripsy system of claim 1 wherein the first and second shockwave techniques of the first and second therapy heads are selected from the group consisting of an electromagnetic therapy head and an electro-hydraulic therapy head.

5. The lithotripsy system of claim 1 wherein the patient treatment region is selected from the group consisting of at least one of a kidney stone, at least one of a gallstone, and at least one of an orthopedic location within the patient that is conducive to fragmentation with a shockwave from one of the therapy heads.

6. The lithotripsy system of claim 1 further comprising an adjustable patient table.

7. The lithotripsy system of claim 1 further comprising a c-arm to adjust alignment of the therapy head.

8. The lithotripsy system of claim 1 wherein a life cycle of the lithotripsy system is increased by software adjustments in electromagnet operations.

9. The lithotripsy system of claim 1 further comprising:
precision mounting brackets to accommodate high voltage connections within the lithotripsy system.

10. The lithotripsy system of claim 1 further comprising an integrated isocentric c-arm to reduce portable equipment requirements.

11. A method for operating a lithotripsy system comprising:
adjusting a first therapy head within the lithotripsy system to provide alignment of the first therapy head with a patient such that a specific target area in a patient treatment region may be located for shockwave treatment using the lithotripsy system with the first therapy head; and
replacing the first therapy head with a second therapy head, wherein the second therapy head is configured to provide shockwave treatment using the lithotripsy system.

12. The method of claim 11 further comprising performing a second shockwave treatment with the second therapy head that is distinct from a first shockwave treatment of the first therapy head.

13. The method of claim 11 wherein said replacing the first therapy head with the second therapy head is performed while a patient remains in close proximity with a patient table of the lithotripsy system.

14. The method of claim 11 further comprising moving a c-arm to adjust alignment of a therapy head.

15. The method of claim 11 further comprising replacing the first therapy head with the second therapy head by mounting the second therapy head with precision mounting brackets to accommodate high voltage connections within the lithotripsy system.

16. The method of claim 11 further comprising adjusting an integrated isocentric c-arm within the lithotripter system.
